(19) United States
(12) Patent Application Publication
(10) Pub. No.: US 2004/0058449 A1
(43) Pub. Date: Mar. 25, 2004

(54) Component analysis of mixtures by nuclear magnetic resonance

(75) Inventors: Thomas Alan Early, Clifton Park, NY (US); Paul Emery Donahue, Niskayuna, NY (US); Elizabeth Anne Williams, Scotia, NY (US)

Correspondence Address: FOLEY AND LARDNER, SUITE 500, 3000 K STREET NW, WASHINGTON, DC, 20007, US

(73) Assignee: GENERAL ELECTRIC COMPANY, Schenectady, NY

(21) Appl. No.: 10/065,163

(22) Filed: Sep. 23, 2002

(51) Int. Cl. G01N 33/00

(52) U.S. Cl. 436/86; 436/173

(57) ABSTRACT

A method has been developed that utilizes NMR integral data from an NMR spectrum for the determination of the concentrations of two or more components in a sample.

### FIELD OF THE INVENTION

The invention relates to a method for determining the concentration of two or more components in a sample using integral data from all resonances in an NMR spectrum. The method emphasizes the incorporation of the complete integral data of an NMR spectrum, regardless of the degree of overlap of different components in the same integral.

### BACKGROUND OF THE INVENTION

An important characteristic of nuclear magnetic resonance (NMR) spectroscopy is that when certain data acquisition and processing conditions are met, NMR spectral intensity from a given sample is proportional to the number of magnetic nuclei in that sample. Combined with the chemical shift effect, which provides a resonance frequency selectivity of the chemical environment of each magnetic nucleus of the sample, this linear relationship of sample quantity and NMR signal intensity allows for quantification of the sample in different ways.

Molecular chemical stoichiometry is reflected in the integral ratios of chemically unique resonances observed in the sample. This has been a powerful tool for small molecule structure determination. Not only is the molecular stoichiometry revealed, but the symmetry of the molecular structure acts to simplify the observed NMR spectra and increase sensitivity. Nuclei such as ¹H, ¹³C, ²⁹Si and ³¹P are useful for this kind of quantitative work because of the relatively high sensitivity and the good to excellent chemical shift resolution.

In a similar fashion, macromolecular characterization via NMR signal integration provides important evidence of the sample stoichiometry. In addition to simple monomeric characterization, NMR of polymers can provide evidence for macromolecular stoichiometry. For example, NMR spectra of copolymeric systems can provide direct quantitative information about polymer composition. However, because of the reduced resolution of spectra of macromolecules (due in part to the slower molecular motion of these systems) quantitative information is more difficult to obtain. This reduction in chemical resolution is particularly noticed in ¹H NMR spectroscopy because of the limited resolution of this nucleus arising from the small chemical shift range (approx. 15 ppm).

Nonetheless, reliable techniques have been reported which utilize those resonances which can be resolved in order to deduce polymer component stoichiometry. Crowded areas of the spectra containing many overlapping resonances from different monomeric constituents are frequently ignored in favor of more easily interpreted areas.

### SUMMARY OF THE INVENTION

In one aspect, the preferred embodiments of the present invention relate to a method for determining relative concentrations of two or more components in a sample comprising using NMR integration values of resonance packets to determine the relative concentrations of two or more components in a sample.

In another aspect, the preferred embodiments of the present invention relate to a method for determining the relative concentrations of two or more components in a sample comprising obtaining a nuclear magnetic resonance spectrum of the sample, identifying resonance packets from the spectrum, integrating the resonance packet, identifying the number of nuclei that contribute to the integral data of the resonance packets and determining the relative concentration of each component in the sample based on the integral data and on the number of nuclei.

### BRIEF DESCRIPTION OF THE DRAWINGS

FIG. 1 is a proton NMR spectrum with integrals of the test mixture of mesitol, diethylphthalate and menthol. This spectrum is divided into nine resonance packets, A through I.

FIG. 2 is a carbon NMR spectrum with integrals of the test mixture of mesitol, diethylphthalate and menthol. This spectrum is divided into eighteen resonance packets, A through R.

### DETAILED DESCRIPTION OF THE PREFERRED EMBODIMENTS

The present inventors realized that peak overlap areas of the NMR spectrum provide useful information about macromolecular stoichiometry in addition to the better resolved areas of the spectrum. The inventors have developed a method that allows the inclusion of these complex data in a straightforward way. The method utilizes a matrix-based calculation that encourages the incorporation of all NMR integral data in order to produce a more accurate interpretation. Another benefit of the method is that a statistical analysis of residual errors of the fit of integral data provide a standard error of analysis, allowing the practitioner a statistical estimate of the errors associated with the calculated concentrations.

An NMR spectrum is made up of a collection of overlapping or nearly overlapping resonances which will be referred to as resonance packets. A useful method of separation of resonance packets is to require baseline resolution between adjacent resonance packets. If attention is given to the details of the data acquisition and processing in order to insure that quantitative spectra are obtained, the integrated intensity of an individual NMR resonance line is linearly proportional to the concentration of nuclei of that resonance frequency in the sample. If molecular symmetry, accidental degeneracy or just similar chemical shifts will result in spectral overlap, then the integral of these resonance packets is proportional to the sum over all components of the products of molecular concentration and the number of nuclei in that resonance packet.

For example, a proton NMR sample containing a solution of only chloroform and benzene in a non-proton containing solvent presents a system in which the analysis of the proton spectrum is separated into two resonance packets with the sole resonance of each component in each of the two packets. The integral of the packet containing the chloroform resonance is proportional to the concentration of chloroform and the integral of the packet containing the benzene resonance is proportional to six times the concentration of benzene. This is because a chloroform molecule contains only one proton while a benzene molecule contains six. However, if one considers a resonance packet to be the entire proton spectrum, then the integral of this packet is proportional to the concentration of chloroform plus six times the concentration of benzene. Then in a complex mixture made up of n molecular components, the different resonance packet integrals are made up of contributions from individual components so that:

$${\sum }_{n} {a}_{m n} {X}_{n} = {b}_{m}$$

where bₘ are the m integrals from each packet. Each packet contains a ₘₙ nuclei from n different components and the concentration of each component is xₙ. To simplify as much as possible, the integral values, bₘ are expressed in arbitrary units. Thus, the expression for all resonance packet integrals for a given spectrum can be expressed as a simple matrix equality.

A x=b [1]

where A is the m by n matrix made up of elements aₘₙ, and x and b are vectors with elements xₙ (the component concentrations) and bₘ (the resonance packet integrals), respectively. As long as there are more observed integrals than individual components (m is greater than n), the concentration of individual components (the vector x) can be calculated.

Problems can arise if two or more different components are distributed identically in the integral packets, i.e., the matrix A has two or more identical columns. In this case, only the sum of concentrations of these two components can be calculated. Likewise, if the integral distribution of a component is a linear combination of the integral distribution of other components, then that component concentration will not be uniquely determined. In these cases, A and x need to be restated in terms of the new, reduced set of x.

When m is greater than n, equation 1 is said to be overdetermined. Solving for x will provide the best values according to the lowest overall least squares difference between the observed and calculated resonance packet integrals. The method used for solving this overdetermined case is to multiply both sides of equation 1 by the transpose of A:

A′A x=A′b. [2]

As long as each column of matrix A is unique and not a linear combination of other columns and m is greater than n, then the matrix product A′A is an n x n matrix symmetric about the diagonal and equation 2 is guaranteed to have a real solution, x. The total standard error, “se,” of equation 2 is:

$$se = \sqrt{\frac{\sum Δ {b}_{m}^{2}}{m - n}}$$

where ^{Δbm} is the difference between the calculated and observed values of the resonance packet integrals. The errors in the calculated component concentrations are proportional to the diagonal elements of the inverse of the matrix A′A, called cofactors, Cᵢᵢ. Normally, a standard error is reported for each component concentration and is se √Cᵢᵢ.

The determination of each component concentration does not require even one resonance packet to be made up of only contributions from that component. That is to say each component concentration, xₙ, will be uniquely determined no matter how their individual resonances are distributed in the chosen resonance packets as long as the distribution is unique for each component.

The methodology discussed above for the determination of each component concentration is not limited to the use of ¹H and ¹³C NMR spectra. In principle, any NMR-active nuclei may be used. Other preferred nuclei that are useful in the practice of the invention are selected from the group consisting of ¹⁵N, ¹⁹F, ²⁹Si, ³¹P, ¹¹ B, ^{O,} ²³Na, ²⁷Al and ²⁹Si. For additional nuclei that may be used see Jeremy K. M. Sanders & Brian K. Hunter, Modern NMR Spectroscopy (2d ed. 1993).

From the foregoing discussion, it is evident that in one aspect, the preferred aspects of the invention relate to a method for determining the relative concentrations of two or more components in a sample. The manner in which this is accomplished is by first obtaining a nuclear magnetic resonance spectrum of the sample. Then, one identifies resonance packets from the spectrum. The resonance packets are then integrated. Once the number of nuclei that contribute to the integral data of the resonance packets are identified one can determine the relative concentration of each component in the sample based on the integral data and on the number of nuclei. While this method is preferably implemented by performing the steps in the order recited, one with ordinary skill in the art would recognize that the method need not be carried in such an order. Also, while the method is preferably performed by using samples dissolved in an NMR solvent, the preferred embodiments of the present invention are not so limited. The skilled artisan would recognize that neat liquid samples as well as solid or gas samples may also be used. In that case one must be sure that the spectra are obtained quantitatively, i.e., the relaxation time between excitation pulses is at least 5 times T₁.

It is evident from the foregoing discussion that the method of the preferred embodiments of the present invention can be implemented to the analysis of complex mixtures. One of the advantages of this method is that the components of such complex mixtures can be quantified without having to physically separate them. For example, monomer content in polymers can be quantified, including possibly rare monomer components like cross-link sites and/or end groups. In addition, the method may be extended to the characterization of polymers which include, but are not limited to, proteins, peptides or polypeptides. In addition, the method may be implemented in schemes for quality assurance/quality control (e.g., impurity detection and quantification).

### EXAMPLES

Test sample. A sample was generated containing three components, 2,4,6-trimethylphenol (mesitol), diethylphthalate and menthol. Samples were purchased from Aldrich and are reported to be >99% pure and were used without further purification. Exactly 0.01 moles of each component was dissolved in 10.0 ml of CDCl3. The density of each solution was then measured by weighing exactly 1.0 ml. To make the NMR test solution, exactly 1.0, 2.0 and 3.0 ml of the mesitol, diethylphthalate and menthol solutions were added together.

TABLE 1
The formulated and NMR-measured mole percentages for the three
component test mixture are given along with the observed molar ratios
according to calculations based on ¹H and ¹³C NMR integrals.

| Component | Mesitol | Diethylphthalate | Menthol |
|---|---|---|---|
| Charged | 17.06 | 32.86 | 50.09 |
| via ¹H NMR | 17.47 | 32.51 | 50.02 |
| via ¹³C NMR | 17.01 | 33.00 | 50.00 |

NMR Measurements and calculations. All NMR spectra were obtained on a General Electric Omega 300WB NMR spectrometer. The proton spectra were obtained in a 5 mm carbon-proton dual probe. About 20 mg of polymer were dissolved in 0.5 ml of CDCl₃. Thirty-two scans were accumulated with 90° pulses, waiting 30 seconds between pulses. The carbon spectra were obtained on a 10 mm broadband probe. About 250 mg of sample along with about 50 mg of Cr(acac)₃ were dissolved in 3.5 ml CDCl₃. About 2000 scans were accumulated with 90° pulses. Gated, broadband ¹H decoupling was applied during the acquisition of the free induction decay. Six scans were acquired per minute.

All spectra were processed and integrated with a customized processing software package. In order to minimize spectral distortion due to finite bandwidth of the spectrometer, spectral widths were adjusted to be about three times the necessary width and the transformed spectra were compensated for the spectrometer filter roll-off by an experimentally determined attenuation curve. Modern spectrometers with digital detection schemes may not need this compensation. Before this filter correction was performed, spectra were baseline corrected with a ninth order (or lower) polynomial fit to user specified regions of the baseline. In this process, the user identifies graphically areas of the spectrum which contain no resonance intensity and every data point identified is least squares fit to the polynomial. This step eliminates the need for highly subjective and arbitrary adjustments of the slope and curvature during the integration process. With moderately well resolved spectra and adequate spectral windows, this baseline correction step is reasonably free from subjective operator judgement and results in highly accurate integrals.

Briefly, the customized software package mentioned above is a PC-based computer program that allows any PC on a network to access, process, analyze and plot NMR spectra from any NMR server on a network. The software is the visible part of a pair of programs that are used for the rapid exchange, processing and plotting of NMR data. Using a dedicated TCP/IP port, the package follows a client-server model for data exchange. The invisible portion of the package, the NMR servers (typically NMR spectrometers and workstations), act as sources of NMR data streams, serving NMR data to clients, anywhere in the world on the network.

The NMR data client-server uses a common data exchange format to facilitate the transportation of data from the source to the destination. The NMR data servers listen on the dedicated NMR TCP/IP port for requests for data or information. An NMR client located anywhere on the network places a request for information to an NMR server using the NMR TCP/IP port. The server receives the request and, after validating the source of the request, formats the information being requested to a common data format. Each NMR server knows how to reformat its native data to the common data format. The data is then sent as TCP/IP packet streams to the requesting client over the NMR port. As the stream is being received, any reformatting necessary to convert the common data format to the native format used on the client is performed by the client. Again the client knows how to convert the common data format to its native format.

Linear regression for the solution of the equation A x=b was done with a custom program utilizing LU matrix decomposition. See Kendall E. Atkinson, “An Introduction to Numerical Analysis”, John Wiley & Sons, 1978, incorporated by reference herein.

Results

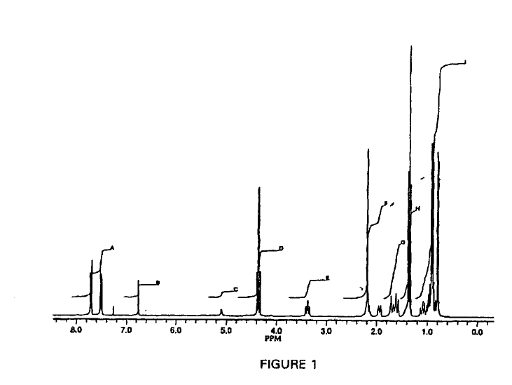

A three component solution, made up from mesitol (2,4,6-trimethylphenol), diethylphthalate and menthol in an approximate 1:2:3 molar ratio, was analyzed. The proton spectrum shown in FIG. 1 was broken up into nine integral packets, A-I. Assignments of the integral packets are described in Table 2 below.

TABLE 2
The matrix A and vector b for the ¹H integrals (shown in FIG. 1) of a
three component mixture of mesitol, diethylphthalate and menthol is
listed.

| Resonance | A | | | b |
|---|---|---|---|---|
| Packet | Mesitol | Diethylphthalate | Menthol | Integral |
| A | 0 | 4 | 0 | 376.3 |
| B | 2 | 0 | 0 | 98.9 |
| C | 1 | 0 | 0 | 47.9 |
| D | 0 | 4 | 0 | 375.4 |
| E | 0 | 0 | 1 | 142.6 |
| F | 9 | 0 | 2 | 740.8 |
| G | 0 | 0 | 3 | 433.4 |
| H | 0 | 6 | 1 | 702.9 |
| I | 0 | 0 | 13 | 1870.7 |

Summing the columns will result in the total number of protons of each of the three components: 12, 14 and 20 for mesitol, diethylphthalate and menthol, respectively. Scanning across the rows of matrix A shows the complexity of each resonance packet. In this case, only two integrals contain resonances from multiple components. In general, polymer component analysis will not be so well partitioned. Best fit mole fractions of the three components based on the above data are listed in Table 1.

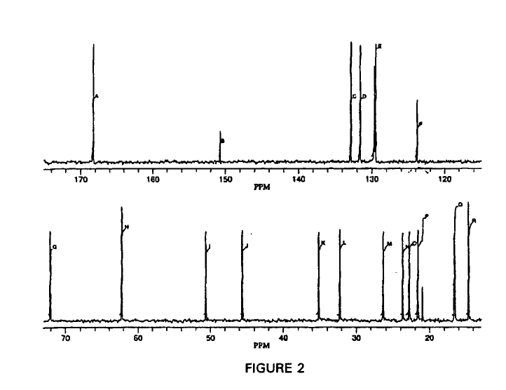

The carbon spectrum was also obtained on the mixture and is shown in FIG. 2. Again, spectral resolution was very good allowing for the separation of each component's resonances. The A and b data for the carbon spectrum are listed in Table 3.

TABLE 3
The matrix A and vector b for the ¹³C integrals (shown in FIG. 2) of a
three component mixture of mesitol, diethylphthalate and menthol is
listed.

| Resonance | A | | | b |
|---|---|---|---|---|
| Packet | Mesitol | Diethylphthalate | Menthol | Integral |
| A | 0 | 2 | 0 | 1218.07 |
| B | 1 | 0 | 0 | 313.57 |
| C | 0 | 2 | 0 | 1152.11 |
| D | 0 | 2 | 0 | 1162.43 |
| E | 3 | 2 | 0 | 2143.04 |
| F | 2 | 0 | 0 | 597.17 |
| G | 0 | 0 | 1 | 909.02 |
| H | 0 | 2 | 0 | 1135.73 |
| I | 0 | 0 | 1 | 864.33 |
| J | 0 | 0 | 1 | 888.02 |
| K | 0 | 0 | 1 | 915.06 |
| L | 0 | 0 | 1 | 935.09 |
| M | 0 | 0 | 1 | 897.56 |
| N | 0 | 0 | 1 | 871.23 |
| O | 0 | 0 | 1 | 935.77 |
| P | 1 | 0 | 1 | 1264.84 |
| Q | 2 | 0 | 1 | 1438.73 |
| R | 0 | 2 | 0 | 1233.84 |

In a likewise manner column sums of A result in the total number of carbon atoms of each component: 9, 12 and 10 for mesitol, diethylphthalate and menthol, respectively. Component mole percentages based on these carbon data are also given in Table 1.

Comparison of the small molecule test results summarized in Table 1 clearly show that both ¹H and ¹³C NMR data can provide accurate integrals for the determination of component concentrations. In both cases the NMR results agree well with the actual component concentrations.

In this small molecule test case spectral resolution was nearly perfect. However, in the case of the lower signal-to-noise carbon spectrum it is instructive to consider what happens if component concentrations were calculated by using only a single integral to measure each component. In this case the menthol concentration which would be calculated would vary by about 8% depending on exactly which menthol resonance integral was used to determine its concentration. Thus, in this case, the preferred method is to average over all resonances of menthol to minimize random error of individual integrals caused by the limited signal-to-noise of the spectrum. In any case, it is better to include and account for integrals from all resonance packets. Confidence in the resulting calculated concentrations are always improved.

A method has been presented for the analysis of component concentrations by NMR integrations. In this method, all integrals from a spectrum are used in a matrix approach that will result in more accurate component concentrations. The method does not require individually resolvable resonances from each component, but only that each component be uniquely distributed in each resonance packet. The method also requires that each nucleus from each component be assigned to one and only one resonance packet. This last requirement can be used as an assignment tool for those cases where expected structures are known but spectral assignments are not. In that case assignment guesses are tested, then modified based on the resulting data fitting errors.

From the foregoing description, one skilled in the art can ascertain the essential characteristics of this invention, and without departing from the spirit and scope thereof, can make various changes and modifications of the invention to adapt it to various usage and conditions without undue experimentation. All patents, patent applications and publications cited herein are incorporated by reference in their entirety.

What is claimed is:

1. A method for determining relative concentrations of two or more components in a sample comprising using NMR integration values of resonance packets to determine the relative concentrations of two or more components in a sample.

2. The method of claim 1, wherein said resonance packets comprise one resonance.

3. The method of claim 1, wherein at least one resonance packet comprises more than one resonance.

4. A method for determining the relative concentrations of two or more components in a sample comprising:
obtaining a nuclear magnetic resonance spectrum of the sample;
identifying resonance packets from the spectrum;
integrating said resonance packets;
identifying the number of nuclei that contribute to the integral data of said resonance packets; and
determining the relative concentration of each component in said sample based on the integral data and on the number of nuclei.

5. The method of claim 4, wherein said resonance packets comprise one resonance.

6. The method of claim 4, wherein at least one resonance packet comprises more than one resonance.

7. The method of claim 4, wherein the steps are carried out in the recited order.

8. The method of claim 4, wherein said nuclei is selected from the group consisting of ¹H, ¹³C, ¹⁵N, ¹⁹F, ²⁹Si, ³¹P, ¹¹B, ¹⁷O, ²³Na, ²⁷Al and ²⁹Si.

9. The method of claim 8, wherein said nuclei is selected from the group consisting of ¹H and ¹³C.

10. The method of claim 4, wherein said determination of the concentration of each component in said sample is performed by linear regression analysis.

11. The method of claim 4, wherein the sample comprises a polymer or mixture of polymers.

12. The method of claim 11, wherein said polymer or mixture of polymers comprises a soft segment BPA polycarbonate.

13. The method of claim 11, wherein said polymer or mixture of polymers comprises a protein, polypeptide or peptide.

14. The method of claim 4, wherein said sample is in solution.

15. The method of claim 4, wherein said sample is in the solid state.

16. The method of claim 4, wherein said method is implemented in a quality assurance process.

17. A method for determining the relative concentrations of two or more components in a sample comprising:
obtaining a nuclear magnetic resonance spectrum of the sample, wherein said sample comprises a polymer or a mixture of polymers;
identifying resonance packets from the spectrum;
integrating said resonance packets;
identifying the number of nuclei that contribute to the integral data of said resonance packets, wherein said nuclei are ¹H or ¹³C; and
determining the relative concentration of each component in said sample based on the integral data and on the number of nuclei.

18. The method of claim 17, wherein said sample is in solution.

19. The method of claim 17, wherein said sample is in the solid state.

20. The method of claim 17, wherein said polymer or mixture of polymers comprises a soft segment BPA polycarbonate.

21. The method of claim 17, wherein said polymer or mixture of polymers comprises a protein, polypeptide or peptide.

22. The method of claim 17, wherein said method is implemented in a quality assurance process.
